(19) United States
(12) Patent Application Publication
(10) Pub. No.: US 2003/0033015 A1
(43) Pub. Date: Feb. 13, 2003

(54) Scleral implants for treatment of presbyopia

(75) Inventors: Stephen Q. Zhou, Irvine, CA (US); Christopher D. Wilcox, Mission Viejo, CA (US); Christine Liau, Irvine, CA (US); Igor Valyunin, Laguna Niguel, CA (US)

Correspondence Address: FROST BROWN TODD LLC, 2200 PNC Center, 201 East Fifth Street, Cincinnati, OH, 45202, US

(73) Assignee: Medennium, Inc.

(21) Appl. No.: 09/906,979

(22) Filed: Jul. 17, 2001

(51) Int. Cl. A61F 2/14

(52) U.S. Cl. 623/6.64; 623/4.1

(57) ABSTRACT

A method for treating presbyopia using a scleral insert is described. The scleral insert is prepared from either (or both) of two specific classes of polymeric materials having both viscous and elastic properties. The first class of polymeric materials has a glass transition temperature (T_{g}) at or below human body temperature (37° C.). The second class of polymeric materials has a melting temperature (Tₘ) at or below human body temperature (37° C.). The implant is stored in a frozen, rigid, elongated state prior to insertion in to the eye. Once it is placed on or within the sclera the insert responds to the increase in temperature, due to the surrounding physiochemical environment whereby it becomes soft and expands to reach its final shape. This implant can be inserted in to the eye through a much smaller incision than is used with conventional scleral implant techniques.

### TECHNICAL FIELD

The present invention relates to methods and articles for treating presbyopia and related conditions of the eye, by using specially designed implants to increase the effective working distance of the ciliary muscle.

### BACKGROUND OF THE INVENTION

In order for the human eye to have clear vision of objects at different distances, the effective focal length of the eye must be adjusted to keep the image of the object focused as sharply as possible on the retina. This change in effective focal length is known as accommodation and is accomplished in the eye by varying the shape of the crystalline lens. Generally, in the unaccommodated emmetropic eye, the curvature of the lens is such that distant objects are sharply imaged on the retina. In the unaccommodated eye, near images are not focused sharply on the retina because their images lie behind the retinal surface. In order to visualize a near object clearly, the curvature of the crystalline lens must be increased, thereby increasing its refractive power and causing the image of the near object to fall on the retina.

The change in shape of the crystalline lens is accomplished by the action of certain muscles and structures within the eyeball or globe of the eye. The lens is located in the forward part of the eye, immediately behind the pupil. It has the shape of a classical biconvex optical lens, i.e., it has a generally circular cross-section of a two-convex-refracting surfaces, and is located generally on the optical axis of the eye, i.e., a straight line drawn from the center of the cornea to the macula in the retina at the posterior portion of the globe. Generally, the curvature of the posterior surface of the lens, i.e., the surface adjacent to the vitreous body, is somewhat greater than that of the anterior surface. The lens is closely surrounded by a membranous capsule that serves as an intermediate structure in the support and actuation of the lens. The lens and its capsule are suspended on the optical axis behind the pupil by a circular assembly of many radially-directed collagenous fibers, the zonules, which are attached at their inner ends to the lens capsule and at their outer ends to the ciliary body, a muscular ring of tissue located just within the outer supporting structure of the eye, the sclera. The ciliary body is relaxed in the unaccommodated eye and therefore assumes its largest diameter. According to the classical theory of accommodation, the relatively large diameter of the ciliary body in this condition causes a tension on the zonules which in turn pull radially outward on the lens capsule, causing the equatorial diameter of the lens to increase slightly, and decreasing the anterior-posterior dimension of the lens at the optical axis. Thus, the tension on the lens capsule causes the lens to assume a flattened state wherein the curvature of the anterior surface, and to some extent of the posterior surface, is less than it would be in the absence of the tension. In this state, the refractive power of the lens is relatively low and the eye is focused for clear vision of distant objects.

When the eye is intended to be focused on a near object, the muscles of the ciliary body contract. This contraction causes the ciliary body to move forward and inward, thereby relaxing the outward pull of the zonules on the equator of the lens capsule. This reduced zonular tension allows the elastic capsule of the lens to contract causing an increase in the anterior-posterior diameter of the lens (i.e., the lens becomes more spherical) resulting in an increase in the optical power of the lens. Because of topographical differences in the thickness of the lens capsule, the central anterior radius of curvature decreases more than the central posterior radius of curvature. This is the accommodated condition of the eye wherein the image of near objects falls sharply on the retina. See Koretz, et al., Scientific American, July, 1988, pages 64-71.

Presbyopia is the decrease in the amplitude of accommodation that is frequently observed in individuals over 40 years of age. In the person having normal vision, i.e., having emmetropic eyes, the ability to focus on near objects is gradually lost, and the individual comes to need glasses for tasks requiring near vision, such as reading. One of the theories for loss of accommodation is that the space between the natural lens and the equatorial ciliary muscle becomes too small as the natural lens continues to grow in diameter in the person of increasing age. Accordingly, one method for treatment of presbyopia is to increase the space between the natural lens and the equatorial ciliary muscle.

Based on this theory, surgical procedures using scleral implants designed for increasing the effective working distance of the ciliary muscle have been developed and clinically tested on human subjects. Examples of such scleral implants, made from hard materials, such as PMMA, or soft materials, such as silicone, include the following: U.S. Pat. No. 6,197,056, Schachar, issued Mar. 6, 2001; U.S. Pat. No. 6,007,578, Schachar, issued Dec. 28, 1999; U.S. Pat. No. 5,722,952, Schachar, issued Mar. 3, 1998; U.S. Pat. No. 5,354,331, Schachar, issued Oct. 11, 1994; U.S. Pat. No. 5,465,737, Schachar, issued Nov. 14, 1995; U.S. Pat. No. 5,489,299, Schachar, issued Feb. 6, 1996; U.S. Pat. No. 5,503,165, Schachar, issued Apr. 2, 1996; U.S. Pat. No. 5,529,076, Schachar, issued Jun. 25, 1996; and Schachar, et al., Annals of Ophthalmology, 1995; 27(2): 58-67; all of which are incorporated herein by reference.

When a conventional scleral expansion band, either made from a hard material (such as PMMA) or a soft material (such as silicone), is implanted into the sclera, the size of the scleral tunnel or the incision on the sclera has to be approximately the same as the implant. Ideally, the incision should be as small as possible in order to help facilitate a fast recovery with a minimized chance of infection. This is difficult to accomplish using the typical materials utilized for scleral implants. It has now been found in the present invention that the use of a thermodynamic material for the preparation of a scleral expansion band permits the insertion of such bands using incisions of minimized size. The bands of the present invention can be pre-stretched and frozen into diameters which are smaller than the dimension in the intended use condition, so that the band can be implanted using a smaller size incision. Upon warming up by the body tissue, the pre-stretched band becomes soft and expands to become larger in diameter and shorter in length, effectively working as a scleral expansion band. Since the implant is a hard solid at room temperature, it is easy to insert; at the same time, since the material becomes soft at body temperature, patient discomfort is minimized. Finally, since the incision size is small, it is possible to use six or eight bands for one eye instead of four bands as is usually used with conventional materials. In this way, the expansion force can be evenly distributed around the circular scleral ring.

U.S. Pat. No. 6,234,175, Zhou, et al., issued May 22, 2001, describes an ocular plug design and a method of insertion of such plugs for the treatment of dry eye. The ocular plug is a narrow rod-like cylinder of appropriate diameter, which is tapered at one end, for insertion into an ocular channel. The plug is prepared from either (or both) of two specific classes of polymeric materials having both viscous and elastic properties. The plug is stored in a frozen, rigid, elongated state prior to insertion into an ocular channel. Once inserted into an ocular channel, the plug responds to an increase in temperature, due to the surrounding physiochemical environment, whereby it becomes soft and the plug subsequently expands to adapt to the size and shape of the patient's punctum or canaliculum. Once the plug expands to the size of the particular ocular channel, the plug is met with a resistance from the surrounding tissue. At that point, expansion of the plug ceases and the plug can effectively block tear drainage through either ocular channel.

### SUMMARY OF THE INVENTION

The present invention relates to a method of increasing the amplitude of accommodation of a human eye having a crystalline lens and a ciliary muscle comprising increasing the effective working distance of the ciliary muscle by increasing the radial distance between the equator of the crystalline lens and the inner diameter of the ciliary muscle by inserting on or within the sclera a prosthetic implant made from a biocompatible composition which:

i. is rigid at room temperature;

ii. becomes elastic when warmed to a temperature above its melting temperature, Tₘ;

iii. becomes rigid again when cooled to a temperature below its Tₘ; and

iv. comprises a material selected from the group consisting of polymeric materials and mixtures of polymeric materials and waxes.

An alternate implant which may be used in the above-described method is one which:

i. is rigid at room temperature;

ii. becomes elastic when warmed to a temperature above its glass transition temperature, T_{g};

iii. becomes rigid again when cooled to a temperature below its T_{g}; and

iv. comprises a material selected from the group consisting of polymeric materials and mixtures of polymeric materials and waxes.

The method is particularly useful for the treatment of presbyopia in the human eye.

In utilizing the method of the present invention, the prosthetic implant is warmed to a temperature at which it becomes elastic; it is formed into dimensions suitable for insertion on or within the sclera; it is allowed to cool and re-solidify in its stretched form; it is inserted in its stretched and rigid form on or within the sclera; and it is allowed to warm to the body's temperature thereby becoming elastic and expanding to its original size and shape (or the size and shape of the scleral channel).

### BRIEF DESCRIPTION OF THE DRAWINGS

FIG. 1 is a schematic depiction of a quartet of scleral implants (1) positioned in the sclera (2) of the eye.

FIG. 2 is a schematic depiction of an alternative way of positioning scleral implants (1) in the sclera (2) of the eye.

FIG. 3 is a perspective view of one embodiment of the present invention suitable for positioning as shown, for example, in either FIG. 1 or FIG. 2.

FIG. 4 is a perspective view of another embodiment of the present invention preferably for positioning similar to that shown in FIG. 1.

FIG. 5 is a perspective view of still another embodiment containing only two segments.

FIG. 6 to FIG. 11 illustrate additional embodiments of the present invention.

### DETAILED DESCRIPTION OF THE INVENTION

According to the present invention, presbyopia is treated by increasing the effective working distance of the ciliary muscle. A number of procedures are available to the surgeon which can accomplish this increase in effective working distance.

A straightforward method of increasing the effective working distance of ciliary muscle is to increase the distance between the equator of the crystalline lens and the inner diameter of the ciliary body in the presbyopic eye. This increased distance restores, at least to some extent, the distance through which the muscles of the ciliary body can contract, and thereby restores their ability to exert force on the lens and change its shape to accomplish accommodation. Any method that increases the radial distance between the lens and the ciliary body can be effective in achieving this end.

The effective working distance of the ciliary muscle can also be increased by shortening the zonules that connect the ciliary muscle to the equator of the crystalline lens. Similarly, procedures that shorten the body of the ciliary muscle itself or move its insertions in the scleral spur and the choroid can be employed to increase the effective distance of the muscle. Finally, procedures that arrest the growth of the lens can stop the steady loss of amplitude of accommodation.

In the practice of the present invention, the lens-ciliary body radial distance is increased by increasing the diameter of the sclera in the region of the ciliary body. This is accomplished by fastening to the sclera (or implanting within the sclera), in that region, a relatively rigid band (made from a specifically-defined material) having a diameter slightly larger than the section of the globe of the eye in the region of the ciliary body. In this way, the sclera in that region is stretched and expanded so that the diameter of the circle describing the intersection of the plane of the ciliary body with the sclera is slightly increased. The ciliary body, located immediately inside the globe and attached to the sclera in this expanded region is thereby also increased in diameter.

Thus, the scleral expansion band of the present invention is adapted for fastening to (or implanting within) the sclera of a human eyeball in the region of the ciliary body. A variety of shapes of the scleral band, including for example, a frustoconical band, a rod circular in cross-section, an expansion band, a wedge or a segment, may all be utilized herein. Examples of such shaped bands include those disclosed in the following US patents, all of which are incorporated herein by reference: U.S. Pat. No. 6,197,056, Schachar, issued Mar. 6, 2001; U.S. Pat. No. 6,007,578, Schachar, issued Dec. 28, 1999; U.S. Pat. No. 5,722,952, Schachar, issued Mar. 3, 1998; U.S. Pat. No. 5,354,331, Schachar, issued Oct. 11, 1994;

U.S. Pat. No. 5,465,737, Schachar, issued Nov. 14, 1995; U.S. Pat. No. 5,489,299, Schachar, issued Feb. 6, 1996; U.S. Pat. No. 5,503,165, Schachar, issued Apr. 2, 1996; and U.S. Pat. No. 5,529,076, Schachar, issued Jun. 25, 1996.

The scleral band may also be made in a plurality of parts that can be assembled prior to use or may be installed separately to form a complete band. The band may be adjustable in circumference. For example, the band may be formed from a strip of material with overlapping ends so that the ends may slide past one another thereby adjusting the circumference of the band.

Unless defined otherwise, all technical terms and scientific terms used herein have the same meaning as commonly understood by one having ordinary skill in the art to which this invention pertains. Although any methods or materials similar or equivalent to those described herein may be used in the practice of the present invention, preferred methods and materials are described herein.

The term “biocompatible” is intended to mean that no acute physiological activity is observed in response to the presence in the body of the material or substance described as possessing such a property. Examples of unacceptable physiological activity would include surface irritation, cellular edema, etc.

The terms “polymer” and “polymeric material” are used interchangeably herein to refer to materials formed by linking atoms or molecules together in a chain to form a longer molecule, i.e., the polymer. The polymers used in the present invention are preferably biologically inert, biocompatible and non-immunogenic. The particularly preferred polymeric materials are biocompatible, non-immunogenic and not subject to substantial degradation under physiological conditions.

The terms “polymer”, “polymer composition”, “polymeric material”, “composition”, and “composite” are interrelated. The terms “polymer composition” and “polymeric material” are used interchangeably and refer to either the polymer or polymeric material itself as defined herein, or a composite, as defined herein. The term “composite” refers to a combination of a polymer with a biologically inert substance that need not qualify as a “polymer”, but may have the special characteristics of having a melting point above body temperature and may have the ability to provide desirable properties to the polymer (such as to toughen or act as a heat sink for the polymer). Examples of these biologically inert substances are waxes, for example, octadecane or oligomeric polyethylenes.

The term “melting point” (Tₘ) of the polymer refers to the temperature at which the peak of the endotherm rise is observed when the temperature is raised through the first order of transition at standard atmospheric conditions. The first order transition is the melting point of the crystalline domains of the polymer. The peak developed in the trace of a differential scanning calorimeter (DSC) analysis experiment has been used to define this transition (see Encyclopedia of Polymer Science and Engineering, 2^{nd} Ed., Vol. 4, pages 482-519).

The term “glass transition temperature” (T_{g}) refers to the temperature at which the amorphous domains of a polymer take on the characteristic properties of the glassy state—brittleness, stiffness and rigidity. At the glass transition temperature, the solid, glassy polymer begins to soften and flow (see Encyclopedia of Polymer Science and Engineering, 2^{nd} Ed., Vol. 7, pages 531-544).

Polymers useful in the present invention are described in U.S. Pat. No. 6,234,175, Zhou, et al., issued May 22, 2001, incorporated herein by reference.

Main chain crystallizable polymers (MCC polymers) are useful for this invention and are well-known. Some of these polymers are commercially available. These are described by Robert W. Lenz, “Organic Chemistry of Synthetic High Polymers”, John Wiley & Sons, New York, 1967, pp. 44-49, incorporated herein by reference. Generally, these polymers are characterized as having crystallizable structures, such as stiff repeating units or stereoregular repeating units, as part of the main polymer chains. The more persistent the crystalline structural units, the higher the degree of crystallinity of the polymer.

Side chain crystallizable polymers (SCC polymers) are also particularly useful for this invention and also are well-known, some of which are commercially available. These polymers are described in J. Polymer Sci.: Macromol. Rev. 8:117-253 (1974), the disclosure of which is incorporated by reference herein. In general, these polymers are characterized as having a crystallizable cluster off to the side of the main backbone and can be made in several configurations, i.e. homopolymers, random copolymers, block copolymers and graft copolymers.

In general, material compositions of the present invention can be divided into two classes. The first class contains at least one component which has a glass transition temperature (T_{g}) at or below human body temperature (37° C.). The second class contains at least one component which has a melting temperature (Tₘ) at or below human body temperature (37° C.). Compositions containing both the first class and second class can also be used for the present invention as long as either (or both) the T_{g} or Tₘ of the mixture is below about 37° C.

The glass transition temperature of a polymer is the temperature above which the polymer is soft and elastic and below which the polymer is hard or glass-like. Examples of suitable T_{g} polymeric materials include, but are not limited to, silicones, acrylic polymers, polyurethanes, hydrocarbon polymers, copolymers of the foregoing, and any combinations thereof. These polymers may be blended with wax-like materials, such as octadecane, or oligomeric polyethylenes to create a composite that contains both rigid, elastic and viscous properties and has a T_{g} at or below 37° C. Preferably, the T_{g}-based polymeric material is an acrylic ester and more preferably it is a copolymer of laurylmethacrylate and methylmethacrylate.

Generally speaking, the T_{g} of a copolymer containing two or more monomers will be dependent on the percentage composition of the monomers. For example poly(methyl methacrylate) (PMMA) has a T_{g} of 105° C. Therefore, it is soft and rubbery, and it can be molded into various shapes above 105° C. At room temperature, however, PMMA is hard and this is due to the short C-1 side chain. This hardness enhances the elasticity of the copolymer and is the driving force for the stretched polymer to return to its initial shape after the temperature increases above its T_{g}. On the other hand, poly(lauryl methacrylate) (PLMA) has a T_{g} of −65° C., and is soft at room temperature, due in part to the C-12 side chain. Thus, a copolymer containing various ratios of PMMA and PLMA can be designed to achieve any T_{g} in the range of -−5° C. to 105° C.

For instance, a copolymer in a molar ratio of 40% lauryl methacrylate and 60% methyl methacrylate has a T_{g} of 19° C. This particular side-chain copolymer has a number of desirable properties for scleral implant design. Because the T_{g} of this copolymer is 19° C., at room temperature it is elastic and can be stretched. When the stretched sample is placed into ice water for about one minute, it remains in the stretched, rigid form as long as the surrounding temperature is maintained below 19° C. However, those skilled in the art realize that the glass transition temperature for a polymer occurs over a temperature range, possibly 10° C. or even larger, rather than a single sharply defined temperature. Also, since this copolymer has C-12 alkyl side chains, there is a high degree of freedom associated with the various rotational perturbations the molecule may undergo. Such a copolymer is superior to the main chain crystallizable polymers as well as crosslinked polymers since these have much more restricted modes of rotational movement. Thus, the flexibility of the C-12 side chain of the LMA component enables this copolymer to readily conform to the shape of the scleral tunnel. The MMA component of this copolymer is relatively hard and elastic. This elasticity is the driving force for the stretched implant to return to its initial shape. Additionally, the LMA/MMA copolymer can be crosslinked using appropriate crosslinkers. Crosslinking further enhances the elastic properties of this copolymer. Finally, this copolymer is an acrylic ester and polymers of this chemical composition have been most widely used in ophthalmic implants because of their long-term stability and biocompatability.

A second class of polymers which can also serve as an ideal material for scleral implant design include those polymers which have a Tₘ lower than about 37° C. The Tₘ of these polymers is a function of the crystalline structure resulting from the nature of the main chain or side chain. The group of Tₘ materials includes, but is not limited to, those compositions which have a crystalline structure based upon one or more side chains which contain at least 10 carbon atoms, or alternatively, any compositions whose crystalline structure is a function of the polymeric main chain structure.

Examples of side chain crystalline materials include, but not limited to, homopolymers or copolymers that contain one or more monomeric units (wherein n=at least 1 monomer unit) having the general formula:

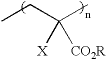

wherein

X is H , or a C₁-C₆ alkyl radical;

R is a linear C₁₀-C₂₆ alkyl radical.

For example, poly(stearylmethacrylate) (PSMA) is a white solid which has an observed melting temperature of 34° C. (see Table 1). This melting temperature is mainly attributed to the crystalline structure of the polymer due to the presence of the pendant 18-carbon side chain. Upon warming the PSMA up to the human body temperature (ca. 37° C.), this white solid is transformed into a clear elastic polymer. Furthermore, the elastic properties of PSMA can be altered by copolymerization with one or more other monomers. Also, whether the PSMA copolymer becomes more elastic or more rigid than PSMA alone is determined by the nature of the added monomers. Table 1 illustrates the properties of various copolymer compositions of stearylmethacrylate (SMA) with methylmethacrylate (MMA). As illustrated in Table 1, when the percentage of MMA increases in the copolymer composition, the copolymer become more rigid and its elasticity increases. For the present invention, preferably this composition is a copolymer constituting at least 95% SMA/5% MMA, and more preferably 97.5% SMA/2.5% MMA.

TABLE 1
SMA/MMA Polymer Compositions and Their
Melting Temperature

| | Weight | Weight | Melting |
|---|---|---|---|
| | of SMA | of MMA | Temperature |
| ID | (grams) | (grams) | (0° C.) |
| PSMA | 1.0 | 0 | 34 |
| 97.5% PSMA | 9.75 | 0.25 | 28 |
| 95% PSMA | 9.50 | 0.50 | 26 |
| 90% PSMA | 9.0 | 1.0 | 22 |
| 80% PSMA | 8.0 | 2.0 | 18 |

(SMA = stearylmethacrylate monomer; MMA = methylmethacrylate monomer)

Examples of the main chain crystallizable materials of the Tₘ family include, but are not limited to, silicone elastomers derived from the general structure of poly[methyl(3,3,3-trifluoro-propyl)siloxane]. Examples of such silicone elastomers are disclosed in U.S. Pat. No. 5,492,993, Saam et al., issued Feb. 20, 1996, and also described in Strain-Induced Crystallization in Poly[methyl(3,3,3-trifluoropropyl)siloxane] Networks, Battjes et al., Macro-molecules, 1995, 28, 790-792, both of which are incorporated by reference herein. As discussed above, it is possible to engineer materials with balanced rigid, elastic and viscous properties. A scleral implant made from these materials is made into an elongated form at temperatures above its T_{g} or Tₘ, and the implant subsequently frozen in its elongated form at temperatures below its T_{g} or Tₘ. Upon insertion into an incision in the sclera, the implant “senses” an increase in its external environmental temperature. In response to this increase in temperature, the elongated rigid implant becomes soft and rubbery, which in turn triggers the shape recovery motion caused by the elastic properties of the implant material. Once the implant approximately corresponds to the size and shape of the scleral channel, resistance from surround tissue stops further expansion of the implant and the implant will “rearrange” itself to the size and shape of the seleral channel based upon the inherent viscosity of the composition. It is noted that such movement by the composition due to its viscosity is at a molecular level and results from the presence from pendant hydrocarbon side chains on the polymer. Thus, the implant is referred to as a “smart implant” since it is able to adapt to the size and shape of the implant channel. Regardless of the particular shape which is utilized, the formed and frozen implant is much smaller in cross section, when inserted into the eye, than it is ultimately when it warms up and takes on the shape of the space where it is placed. This allows the implant procedure to be performed using a much smaller incision than in standard scleral implant techniques. The scleral implant may, for example, take the shape of a ring or band which is very narrow or thin in cross section when inserted but which expands to fill the scleral channel which has been prepared for its implantation. This expansion provides the required spacing which acts to increase the effective working distance of the ciliary muscle. As an alternative to the ring or rod structure, the implants may be in the form of small wedges which are placed at various points in the sclera. Since the incision size is so small, it is possible to use six or eight inserts for one eye, instead of the four bands which are generally used in conventional techniques. In this way, the expansion force is evenly distributed around the circular scleral ring.

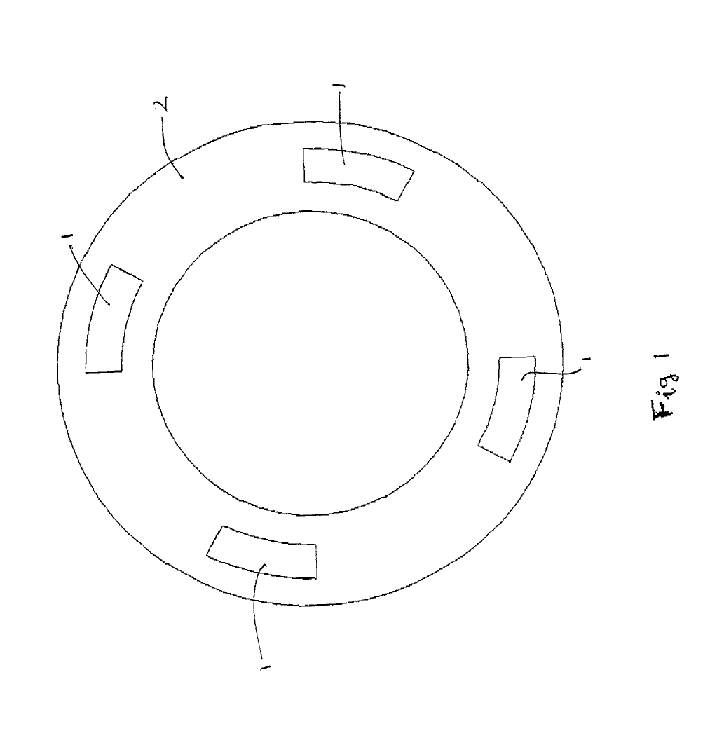

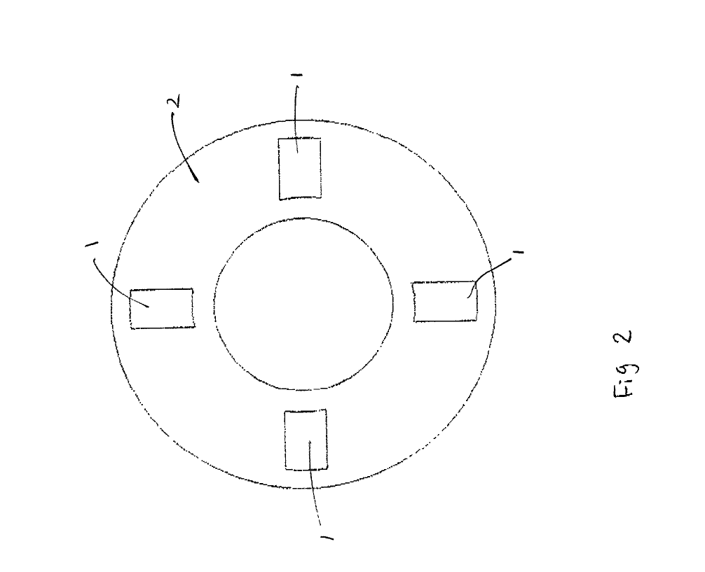

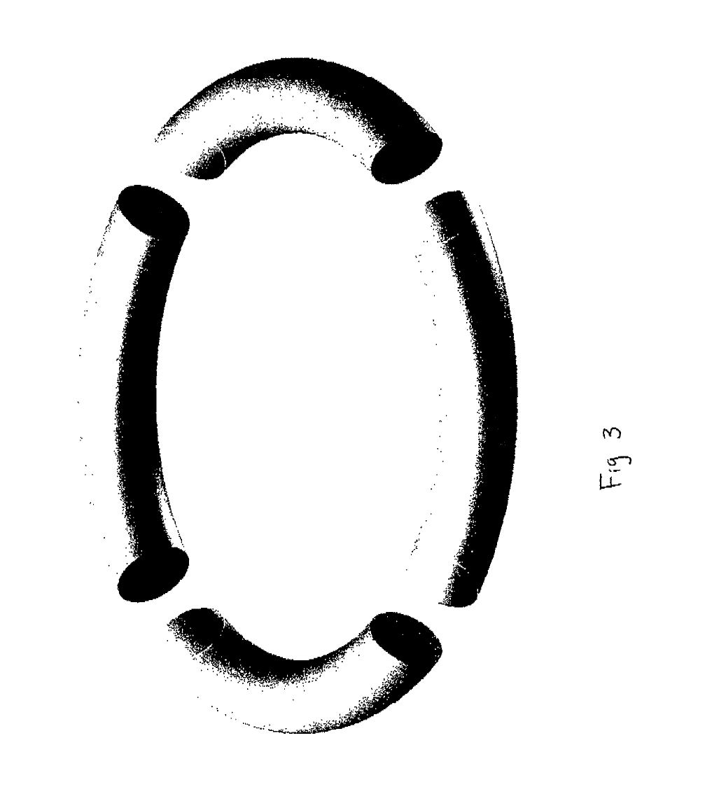

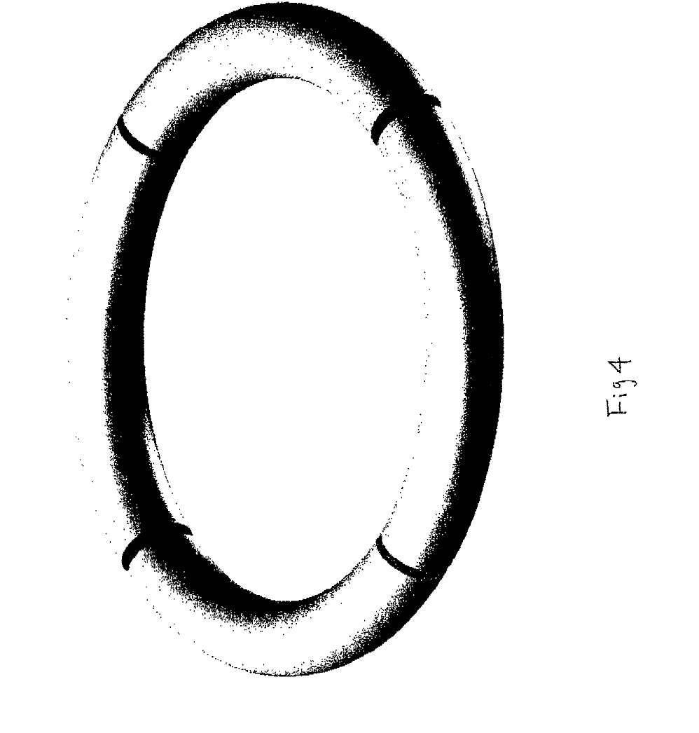

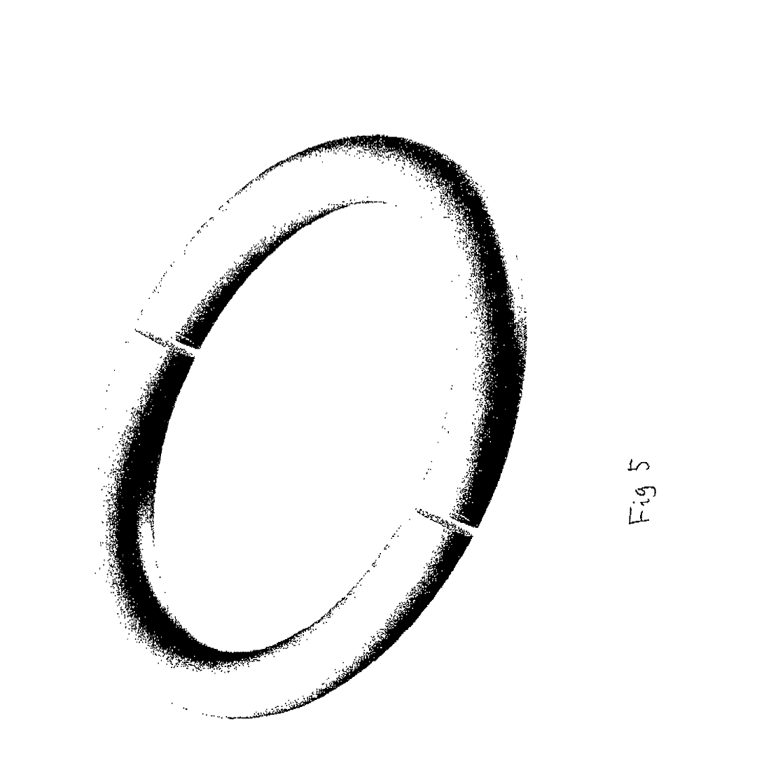

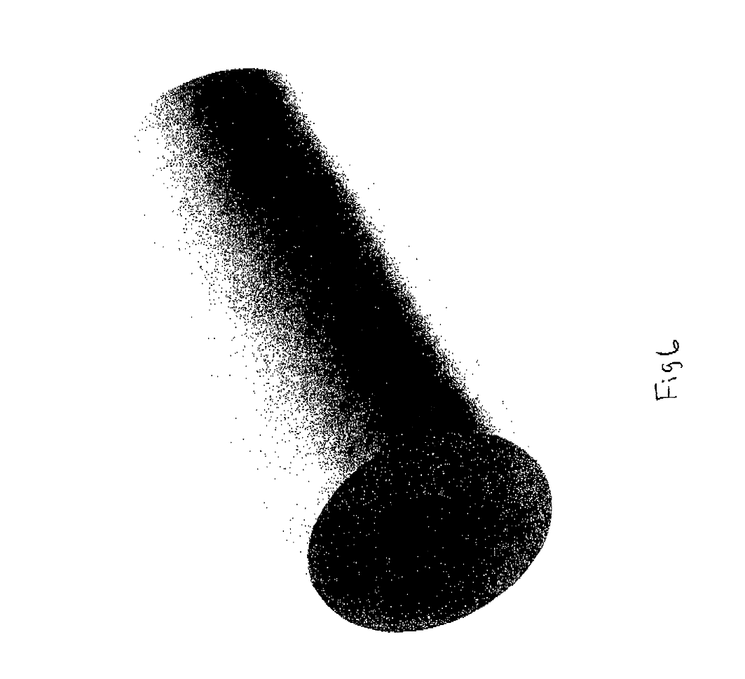

Generally, the scleral implant surgery will involve a partial scleral thickness radial incision in the area about 2 mm posterior of the limbus (sometimes referred to herein as “the scleral channel”). The incision size is in the range of about 1 mm. If a hard scleral (prior art) implant is used, the incision will have to be widened to about 2 mm or more in order to allow the hard implant to be inserted. However, with the scleral implant of the present invention, the widening of the incision opening is not necessary because of the reduced intersection area of the implant provided by the present invention. The positions for scleral implant placement can be, for example, the quartet shown in FIG. 1 or FIG. 2. It is important to note that FIG. 1 and FIG. 2 are only two examples used for the purpose of illustration. It is possible to have other positions for the scleral implants, such as an enclosed ring as shown in FIG. 4 and FIG. 5. It is also possible to use six or even eight smaller implants for one eye.

The scleral implants of the present invention may be made in various shapes, such as cylindrical, wedge, ellipsoid, and oval. The size of the implant depends on patient conditions and the number of implants used for each eye. For example, if eight implants are used instead of four, the size for each implant necessary for achieving sufficient expansion of the scleral perimeter will be smaller than its four-implant counterpart. Generally, the scleral implant has a diameter in the range of from about 0.5 mm to about 3 mm and a length of from about 2 mm to about 8 mm. In some cases, the length can be as long as 20 mm (FIG. 5). In addition, the implants may have a radius so that they conform to the sclera curvature. While other anatomically compatible shapes and dimensions are all possible, the present invention utilizes the thermodynamic properties of the implant material so that they can be inserted through a small incision; that fact may have an impact on the shape of the implant. In its stretched form, the implant typically has a length of from about 8 mm to about 35 mm, and a diameter of from about 0.3 mm to about 2 mm (preferably less than about 1 mm).

In order that the present invention may be more fully understood, the following example is provided by way of illustration only and is not intended to be limiting.

### EXAMPLE

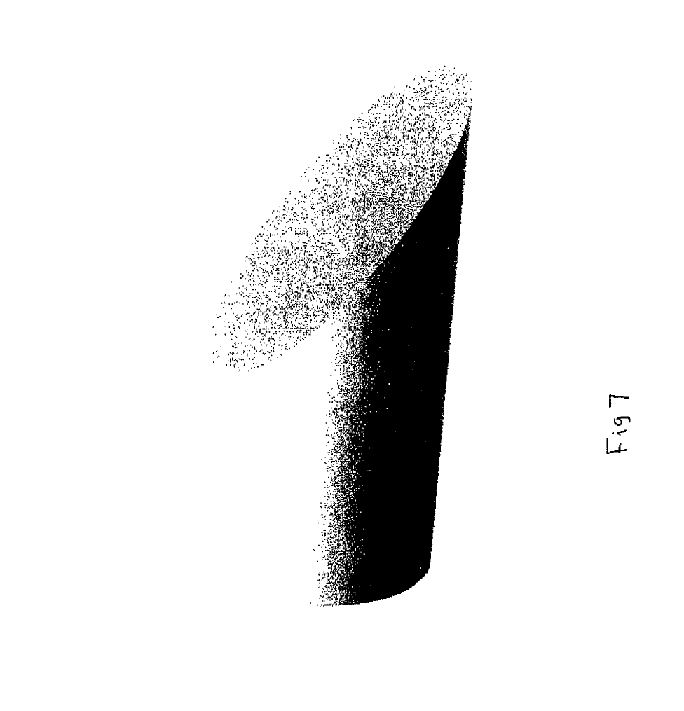

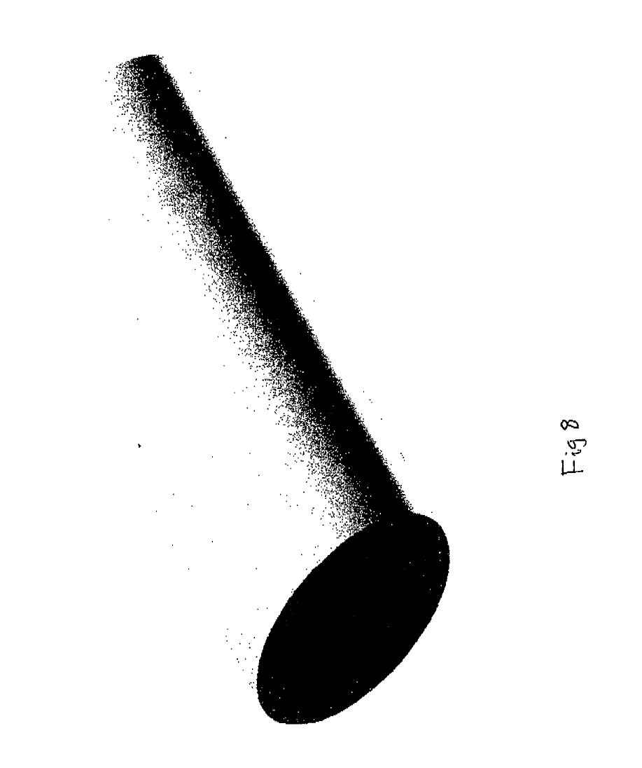

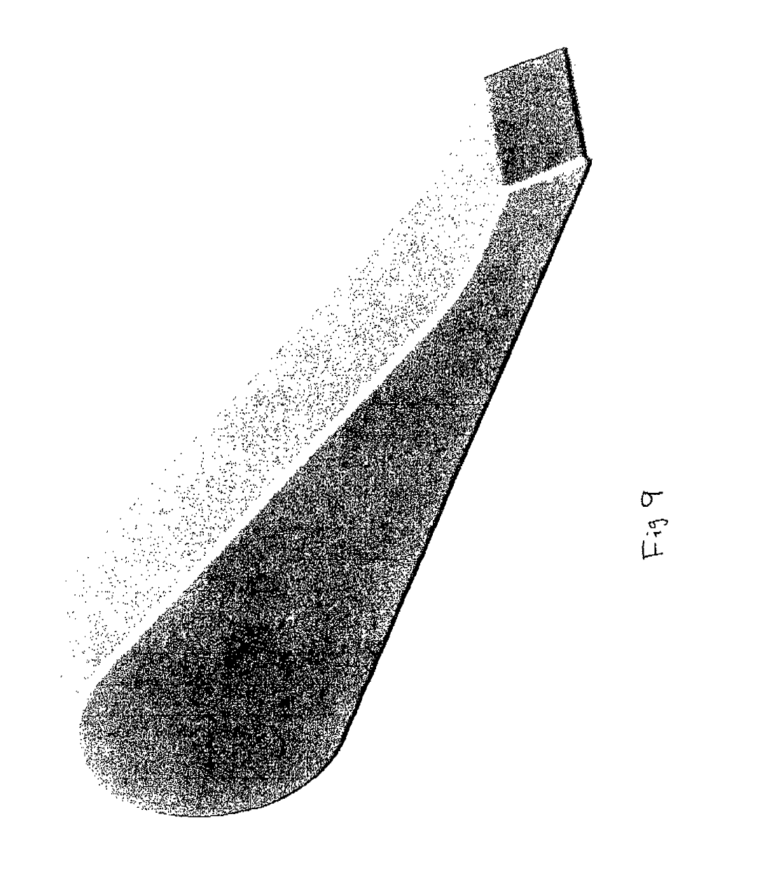

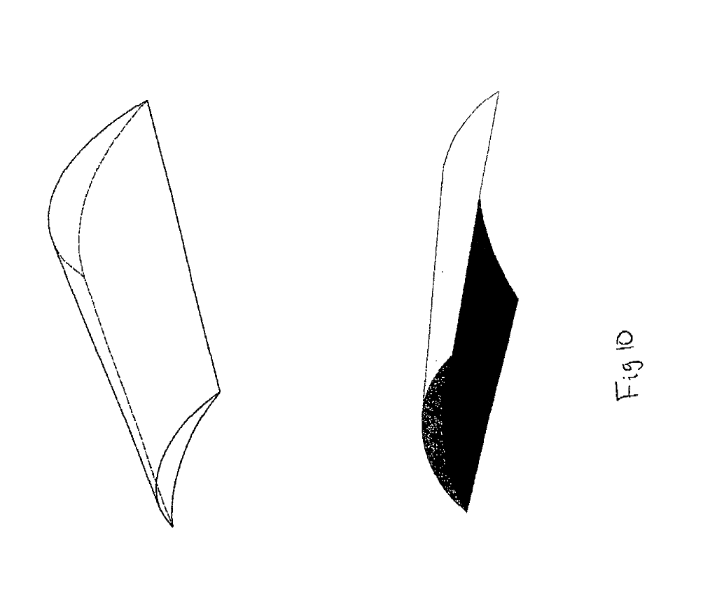

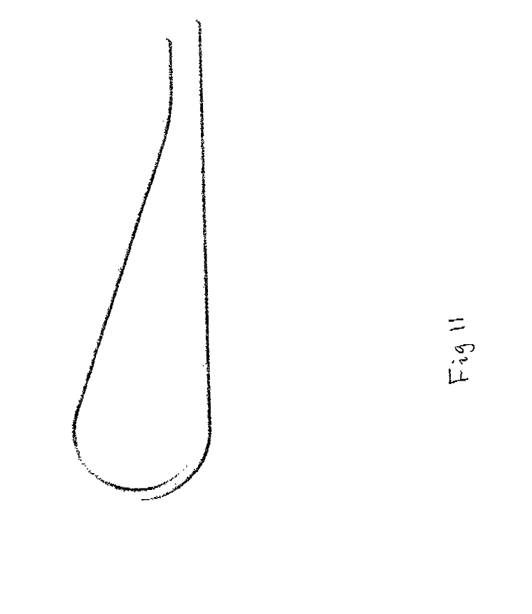

A polypropylene tube with a diameter of 1.5 mm and a length of about 4 inches, and with one end being pre-sealed thermally, was filled with stearyl methacrylate monomer solution with benzoyl peroxide. After the tube was properly sealed and the polymerization reaction completed by heating at 110° C. for about 16 hours, a cylindrical white solid rod of poly(stearyl methacrylate) was obtained. The rod has a diameter of about 1.5 mm and a length of about 2 inches (5.08 cm). This rigid rod can be warmed in a water bath at a temperature of about 50° C. for about one minute, then stretched into a thin rod with a diameter of about 0.5 mm. This thin long rod was then cut into pieces having various lengths, such as 25 mm. If a tapered end is desirable, a slant cut may be performed (see FIG. 7, for example). After gamma-sterilization, the finished small piece of rod may be used as a scleral implant of the present invention.

Because of the reduced diameter from 1.5 mm to 0.5 mm, this implant can be inserted through an incision with a size less than 1 mm. Upon warming up by the body temperature, the implant will become soft and shape recovery will start. Its diameter increases up to 1.5 mm and its length decreases from about 25 mm to about 5 mm.

What is claimed is:

1. A method of increasing the amplitude of accommodation of a human eye, having a crystalline lens and ciliary muscle by increasing the radial distance between the equator of the crystalline lens and the inner diameter of the ciliary muscle by inserting on or within the sclera a prosthetic implant made from a bio-compatible material which:
(i) is rigid at room temperature;
(ii) becomes elastic when warmed to a temperature above its melting temperature, Tₘ;
(iii) becomes rigid again when cooled to a temperature below its Tₘ; and
(iv) comprises a material selected from the group consisting of polymeric materials and mixtures of polymeric materials and waxes.

2. A method of treating presbyopia in a human eye having a crystalline lens and ciliary muscle comprising increasing the effective working distance of the ciliary muscle by increasing the radial distance between the equator of the crystalline lens and the inner diameter of the ciliary muscle by inserting on or within the sclera a prosthetic implant made from a bio-compatible material which:
(i) is rigid at room temperature;
(ii) becomes elastic when warmed to a temperature above its melting temperature, Tₘ;
(iii) becomes rigid again when cooled to a temperature below its Tₘ; and
(iv) comprises a material selected from the group consisting of polymeric materials and mixtures of polymeric materials and waxes.

3. The method according to claim 2 wherein said scleral expansion is accomplished by inserting the said prosthetic implant into a channel beneath the sclera in the region of the ciliary body, said implant having a diameter greater than the interior diameter of the sclera in said region.

4. The method according to claim 2 wherein the scleral implant is processed according to the following steps:
a) prior to implantation, warming said implant to a temperature at which it becomes elastic;
b) forming said implant into dimensions suitable for insertion into a scleral channel;
c) allowing the implant to cool and resolidify in its stretched form;
d) inserting the stretched, rigid implant into said scleral channel; and
e) allowing said implant to warm to the body's temperature, thereby becoming elastic and conforming to the shape of the scleral channel.

5. The method of claim 4 wherein said implant conforms to the shape of the scleral channel in from about one second to about 120 seconds after it has been inserted.

6. The method according to claim 5 wherein said polymeric material is selected from the group consisting of polymers, homopolymers, cross-linked polymers and copolymers of acrylic esters, silicone elastomers, and combinations thereof.

7. The method according to claim 6 wherein said implant has a Tₘ of less than about 37° C.

8. The method according to claim 5 wherein said polymeric material is a side chain crystallizable polymer which comprises an acrylic ester of the formula: 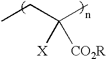
wherein
X is H, or a C₁-C₆ alkyl radical; and
R is a linear C₁₀-C₂₆ alkyl radical.

9. The method according to claim 5 wherein said polymeric material is a main chain crystallizable polymer comprising the silicone elastomer stereo-regular poly[methyl (3,3,3-trifluoropropyl) siloxane].

10. The method according to claim 5 wherein said polymeric material in its stretched form is in the shape of a cylindrical rod-shaped implant which is tapered at one end to facilitate insertion into the scleral channel.

11. The method according to claim 10 wherein said implant has a length of from about 2 mm to about 8 mm and a diameter of from about 0.5 mm to about 3 mm after the shape recovery.

12. The method according to claim 10 wherein said implant in its stretched form has a length of from about 8 mm to about 35 mm and a diameter of from about 0.3 mm to about 2 mm.

13. The method according to claim 12 wherein said polymeric material comprises poly(stearyl methacrylate).

14. A method for increasing the amplitude of accommodation of a human eye having a crystalline lens and ciliary muscle comprising increasing the effective working distance of the ciliary muscle by increasing the radial distance between the equator of the crystalline lens and the inner diameter of the ciliary muscle by inserting on or within the sclera a prosthetic implant made from a bio-compatible material which:
(i) is rigid at room temperature;
(ii) becomes elastic when warmed to a temperature above its glass transition temperature, T_{g};
(iii) becomes rigid again when cooled to a temperature below its T_{g}; and
(iv) comprises a material selected from the group consisting of polymeric materials and mixtures of polymeric materials and waxes.

15. A method of treating presbyopia in a human eye having a crystalline lens and ciliary muscle comprising increasing the effective working distance of the ciliary muscle by increasing the radial distance between the equator of the crystalline lens and the inner diameter of the ciliary muscle by inserting on or within the sclera prosthetic implant made from a bio-compatible material which:
(i) is rigid at room temperature;
(ii) becomes elastic when warmed to a temperature above its glass transition temperature, T_{g};
(iii) becomes rigid again when cooled to a temperature below its T_{g}; and
(iv) comprises a material selected from the group consisting of polymeric materials and mixtures of polymeric materials and waxes.

16. The method according to claim 15 wherein the said scleral expansion is accomplished by inserting into a channel beneath the sclera in the region of the ciliary body said prosthetic implant having a diameter greater than the interior diameter of the sclera in said region.

17. The method according to claim 16 wherein prosthetic implant is processed according to the following steps:
a) prior to insertion, warming said implant to a temperature at which it becomes elastic;
b) forming said implant into dimensions suitable for insertion into a scleral channel;
c) allowing said composition to cool and re-solidify in its de-formed state;
d) inserting said stretched, rigid implant into said scleral channel; and
e) allowing said implant to warm to the body's temperature thereby becoming elastic and conforming to the shape of the scleral channel.

18. The method according to claim 17 wherein the implant fills and includes the scleral channel in about one minute to about seven minutes after it has been inserted.

19. The method according to claim 18 wherein said polymeric material is selected from the group consisting of polymers, homopolymers, cross-linked polymers and copolymers of silicones, acrylic esters, polyurethanes, hydrocarbon polymers and combinations thereof.

20. The method according to claim 17 wherein said implant has a T_{g} of less than about 37° C.

21. The method according to claim 17 wherein said polymeric material is an acrylic ester.

22. The method according to claim 17 wherein said polymeric material in its stretched form is in the shape of a cylindrical rod-shaped implant which is tapered at one end to facilitate insertion into the scleral channel.

23. The method according to claim 17 wherein said implant has a length of from about 2 mm to about 8 mm and a diameter of from about 0.5 mm to about 3 μm after shape recovery.

24. The method according to claim 17 wherein said implant in its stretched form has a length of from about 8 mm to about 35 mm and a diameter of from about 0.3 mm to about 2 mm.

25. The method according to claim 17 wherein said implant is comprised of a polymer of polymethylmethacrylate and polylaurylmethacrylate.
